(19) United States
(12) Patent Application Publication
(10) Pub. No.: US 2011/0150762 A1
(43) Pub. Date: Jun. 23, 2011

(54) BONE CEMENT CONTAINING BONE MARROW

(76) Inventors: Andreas Boger, Oberdorf (CH); Daniel Arens, Oberdorf (CH); Markus Windolf, Oberdorf (CH); Armando Gisep, Marbach (CH)

(21) Appl. No.: 12/868,237

(22) Filed: Aug. 25, 2010

Related U.S. Application Data
(60) Provisional application No. 61/238,870, filed on Sep. 1, 2009.

(51) Int. Cl. A61K 51/00; A61K 35/28

(52) U.S. Cl. 424/1.61; 424/577

(57) ABSTRACT

A bone cement configured to be introduced to a target bone location and allowed to cure includes at least a monomer, and an amount of bone marrow. The resulting cured bone cement includes at least one desired mechanical property that can be tailored to match a like mechanical property of the target bone. The mechanical property can be a material stiffness (Young's modulus) or yield strength.

### CROSS-REFERENCE TO RELATED APPLICATIONS

This application claims priority to U.S. Provisional Patent Application Ser. No. 61/238,870, filed Sep. 1, 2009, the disclosure of which is hereby incorporated by reference as if set forth in its entirety herein.

### BACKGROUND

A number of bone procedures utilise bone cement. For instance, in a bone procedure known as vertebroplasty, vertebral compression fractures in osteoporotic patients are treated by augmenting the fractured vertebral body with a bone cement. The bone cement polymerizes and hardens upon injection into the vertebral body and stabilises the fracture. Pain relief for the patient is usually immediate and vertebroplasty procedures are characterized by a high rate of success.

In one typical example, the bone cement is prepared directly prior to injection by mixing bone cement powder (e.g., poly-methyl-methacrylate (PMMA)), a liquid monomer (e.g., methyl-methacrylate monomer (MMA)), an x-ray contrast agent (e.g., barium sulfate), and an activator of the polymerization reaction (e.g., N, N-dimethyl-p-toluidine) to form a fluid mixture. Other additives including but not limited to stabilizers, drugs, fillers, dyes and fibers may also be included in the bone cement. Since the components react upon mixing, immediately leading to the polymerization, the components of bone cement are kept separate from each other until the user is ready to form the desired bone cement. Once mixed, the user works very quickly because the bone cement sets and hardens rapidly.

Bone cements have a number of mechanical properties that differ from normal bone. For example, the elastic moduli of typical PMMA bone cements lie around 2-3 GPa, while the elastic modulus of osteoporotic cancellous bone lies in the range of 0.1-0.5 GPa. The mismatch in stiffness between the bone cement and the osteoporotic cancellous bone is generally perceived as favoring the subsequent fracturing of bone adjacent to bone cement after completion of the procedure. For instance, the vertebral bodies that are adjacent to the augmented vertebral body may be more prone to fracture after the augmentation procedure. Bone cement with one or more properties that more closely match surrounding bone is desirable.

### SUMMARY

In accordance with one embodiment, a cement is configured to be introduced to a target location and allowed to cure. The cement includes a monomer, a polymerization initiator, and a quantity of bone marrow in sufficient amount such that the cement has a mechanical property that matches a like mechanical property of the target location.

### BRIEF DESCRIPTION OF THE DRAWINGS

The foregoing summary, as well as the following detailed description of an example embodiment of the application, will be better understood when read in conjunction with the appended drawings, in which there is shown in the drawings an example embodiment for the purposes of illustration. It should be understood, however, that the application is not limited to the precise arrangements and instrumentalities shown. In the drawings:

FIG. 1 is a flowchart illustrating a method of fabricating bone cement in accordance with one embodiment;

FIG. 2 is a schematic elevation view of the bone cement fabricated in accordance with the method illustrated in FIG. 1;

FIG. 3 is a side elevation view of a delivery system configured to deliver the bone cement illustrated in FIG. 2 to a target location;

FIG. 4A is a perspective view of four test samples of the bone cement of the type illustrated in FIG. 2;

FIG. 4B is a side elevation view of the four test samples illustrated in FIG. 4A; and

FIG. 5 is a graph plotting cement viscosity as function of time during curing for each of the test samples illustrated in FIGS. 4A-B.

### DETAILED DESCRIPTION

The following detailed description includes references to the accompanying drawings, which form a part of the detailed description. The drawings show, by way of illustration, various embodiments. These embodiments are also referred to herein as “examples.” Such examples can include elements in addition to those shown and described. However, examples are also contemplated in which only those elements shown and described are provided. In the drawings, like numerals describe substantially similar components throughout the several views.

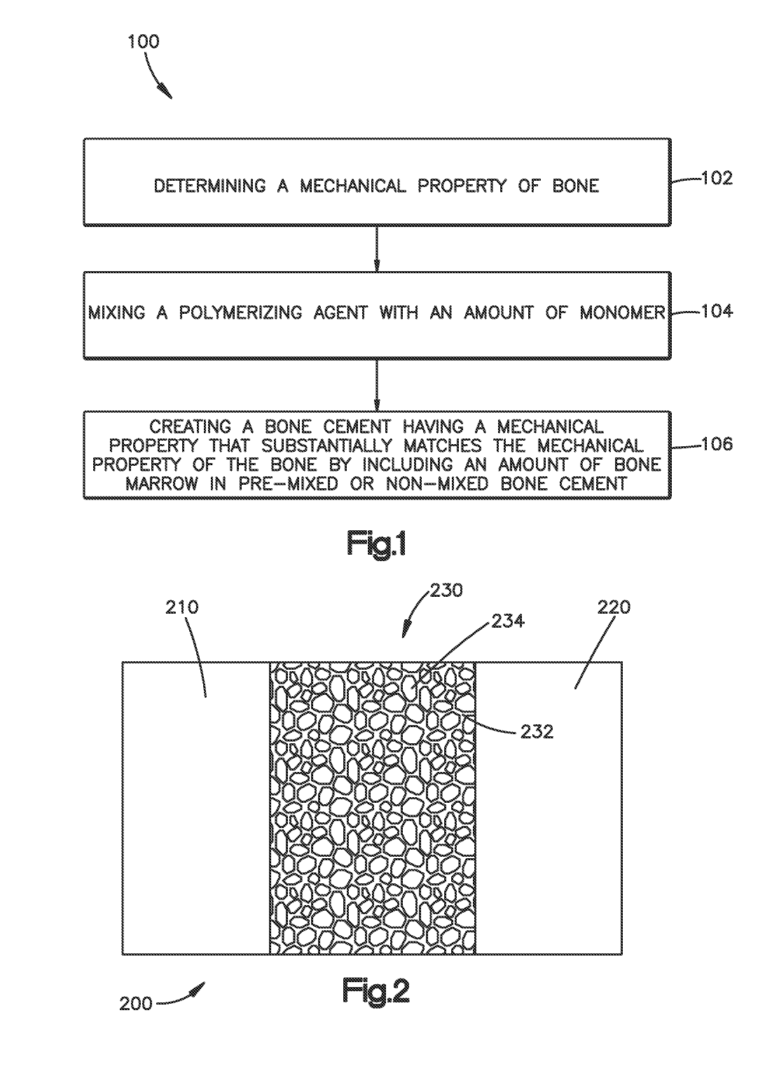

Referring to FIGS. 1-2, a method 100 of fabricating a bone cement 230 in accordance with one embodiment includes the step 102 of determining at least one select mechanical property of a bone to which a like mechanical property of the cement 230 is to be substantially matched. In one example, the mechanical property of the bone cement 230 and the bone is a yield strength. In another example, the mechanical property of the cement 230 and the bone is a Young's modulus or material stiffness. Thus, step 102 can include the step of determining a yield strength of a bone that is to be matched with the cement 230. The step 102 can alternatively or additionally include the step of determining a Young's modulus of the bone that is to be matched with the cement. Thus, the mechanical properties such as yield strength and Young's modulus may or may not be exclusive, and multiple mechanical properties can be chosen for matching at the same time. It should be appreciated that the bone can define a target location for the cement when injected, for instance when augmenting a fracture in the bone.

It is recognized that the cortical bone portion has mechanical properties that differ from the cancellous bone portion. However, due to the small thickness of the cortical bone portion relative to the cancellous bone portion, the mechanical properties bone cement are configured to substantially match those of the cancellous bone portion. The yield strength of cancellous bone can, in some instances, be between approximately 1 MPa and 10 MPa. The Young's modulus of cancellous bone can, in some instances, be between approximately 50 MPa and 1000 MPa. Although example numbers are given, a number of factors in bone provide for different ranges of mechanical properties. For example, a condition of the bone such as an amount of osteoporosity may alter the mechanical property of a given bone. As a result, the ranges given are only examples.

At step 104, a bone cement powder is created by mixing a polymerization initiator (or polymerizing agent) with a desired amount of monomer. In one example, the polymerization initiator can include benzoyl peroxide, although it should be appreciated that any suitable alternative polymerization initiator could be used. The monomer can include a methyl methacrylate monomer. The polymerization initiator can be mixed with the monomer to form a bone cement powder, which can be a polymer such as poly methyl methacrylate (PMMA).

It should be appreciated that the bone cement can be formed from a polymer in the form of substantially PMMA. For instance, the PMMA can be produced in the manner described above, or can further can be a derivative PMMA having one or more styrene groups in the polymer backbone. For instance, the monomer can include styrene groups that build into the polymer backbone. While the polymer is PMMA in accordance with the illustrated embodiment, it should be appreciated that the bone cement can comprise any polymer suitable for forming a reliable bone cement in the presence of bone marrow.

At step 106, the bone cement is created having at least one select mechanical property that substantially matches a corresponding select mechanical property of the target bone. In one example, the yield strength (or compressive yield strength) of the bone cement can be between approximately 5 MPa and approximately 60 MPa, for instance between approximately 5 MPa and approximately 60 MPa, such as between approximately 20 MPa and approximately 50 MPa, and further still between 12 MPa and approximately 25 MPa. In another example, when cured, the bone cement can have a Young's modulus between approximately 50 MPa and approximately 1500 MPa, for instance between approximately 100 MPa and approximately 1000 MPa, such as between approximately 200 MPa and approximately 500 MPa.

It is recognized that the mechanical property of the bone cement can be said to substantially match the like mechanical property of the cancellous bone portion of the target bone (or target bone), even though it is not the same as that of the cancellous bone portion of the target bone (or target bone). For instance, it is believed that providing the mechanical property of the bone cement at a level that more closely matches that of the target bone with respect to conventional bone cements will result in a reduction of the risk of subsequent fracturing of bone adjacent to bone cement after completion of the surgical procedure. Thus, it can be said that the mechanical property (e.g., Young's modulus and/or yield strength) of the bone cement substantially matches that of the target bone, or cancellous portion of the target bone.

It should be appreciated that the bone marrow-containing bone cement can be created at step 106 by first creating a bone cement, and then adding a desired quantity of bone marrow so as to modify the mechanical property of the bone cement to substantially match the like mechanical property of the target bone. For instance, the bone cement powder can be mixed with a monomer, an activator, and a polymerization initiator as desired that creates a bone cement paste. The desired quantity of bone marrow can then be added to the bone cement paste before the bone cement paste cures, thereby modifying the mechanical property of the paste from a first mismatched configuration to a second substantially matched configuration. It should be appreciated that the bone marrow can be added to any one up to all of the bone powder, monomer, activator, and/or the polymerization initiator, either prior to combination with any of the others, alone, or after combination with any of the others.

For instance, at step 106, it is envisioned that a desired amount of bone marrow can be added to the bone cement powder prior to creating the bone cement paste. The mixture of cement powder and bone marrow can then be mixed with the monomer, activator, and polymerization initiator to create a bone cement paste that subsequently cures. Thus, rather than modifying the mechanical properties of a pre-existing bone cement paste by adding the bone marrow to the pre-existing bone paste whose mechanical property is initially mismatched with respect to the like mechanical property of the target bone, it is envisioned that a bone cement paste can be created having the substantially matched mechanical property by adding the bone marrow to the bone cement powder.

Thus, in one example, the bone marrow is included in a particulate component of bone cement, such as the bone cement powder. In another example, bone marrow is added independently of other particulate components after the bone cement powder has been mixed with a monomer (in combination with an activator and/or a polymerization initiator if desired), but before the bone cement has been allowed to cure.

It should thus be appreciated that bone marrow can be added to at least one component of the bone cement prior to curing of the bone cement. The bone marrow can be added to a solid phase of the bone cement (for instance the bone cement powder), or can be added to the liquid phase of the bone cement (for instance including a monomer). Thus, the component to which the bone marrow is added can be a bone cement powder, such as PMMA, a monomer, such as MMA, an activator, a polymerization initiator, or any combination of some or all of the above. It should be further appreciated that different quantities can be included in the bone cement such that the desired mechanical property or properties of the bone cement can be tailored to match the bone into which the bone cement is to be injected, it being appreciated that a bone of a given patient may exhibit a different stiffness or Young's modulus, or yield strength than other bones or other patients, or other bones within the same patient.

In accordance with one embodiment, the bone marrow can be added in a desired amount such that the resulting bone cement contains 10-60% bone marrow by volume. For instance, the bone cement can contain approximately 35% bone marrow by volume.

Referring now to FIG. 2, the bone cement 230 can be used to join a pair of schematically illustrated solid bodies, which can be target bone portions. For instance, a first solid body 210 and a second solid body 220 are shown joined by the bone cement 230. The example bone cement 230 includes a polymer 232 and a particulate component 234. In the example shown, the particulate component 234 is dispersed within the polymer matrix 232.

In one embodiment, the polymer 232 includes PMMA as described above, though it should be appreciated that the polymer 232 can include other polymer chemistries. In one embodiment, the liquid phase of the bone cement, prior to curing, includes an amount of an activator in addition to monomer. One example of an activator includes an amount of N,N-dimethyl-p-toluidine. In one example, the liquid phase can include approximately 97.6 volume percent methyl methacrylate (MMA) monomer and 2.4 volume percent N, N-dimethyl-p-toluidine. Alternatively or additionally, an amount of stabilizer, such as hydroquinone, can be added to the liquid phase. In accordance with one embodiment, the stabilizer can be hydroquinone provided in approximately 20 ppm.

In accordance with one embodiment, the particulate component 234 includes a powder, which can include beads or like structure of PMMA. The addition of the powder component 234 to the polymer matrix 232 can impart a desired viscosity to the bone cement 230 prior to curing.

In selected embodiments, in addition to PMMA, the particulate component 234 further includes the polymerization initiator. In one example, the polymerization initiator includes benzoyl peroxide. In selected embodiments, the particulate component 234 further includes a radiopaque agent, which can include barium sulphate or zirconium dioxide in one example, though it should be appreciated that the particulate component 234 can include other radiopaque agents. For instance, the particulate component 234 can further include a desired amount of hydroxyapatite.

As described above, in one example the bone cement 230 is applied in a non-solid state to a target location, such as between the first solid body 210 and the second solid body 220. In one embodiment, the solid bodies 210 and 220 are bone portions, such as existing separated bone portions, or vertebral bodies that are disposed adjacent augmented vertebral bodies. Alternatively, the solid bodies 210 and 220 can be hardware in the form of implants that are affixed to osteoporotic or cancellous bone.

It should also be recognized that the bone cement 230 can include a predetermined amount of bone marrow that is calculated to substantially match a mechanical property of the target bone for a broad range of patients. Alternatively, the bone cement 230 can be customized to include an amount of bone marrow, determined on a patient-by-patient basis based on the mechanical property of the target bone. For instance, the mechanical property of the target bone of a given patient can be derived based on Computed Tomography (CT) data, and/or using a bone density measuring device, such as Densiprobe™ diagnostic device, developed by the AO Research Institute Davos, located in Davos, Switzerland.

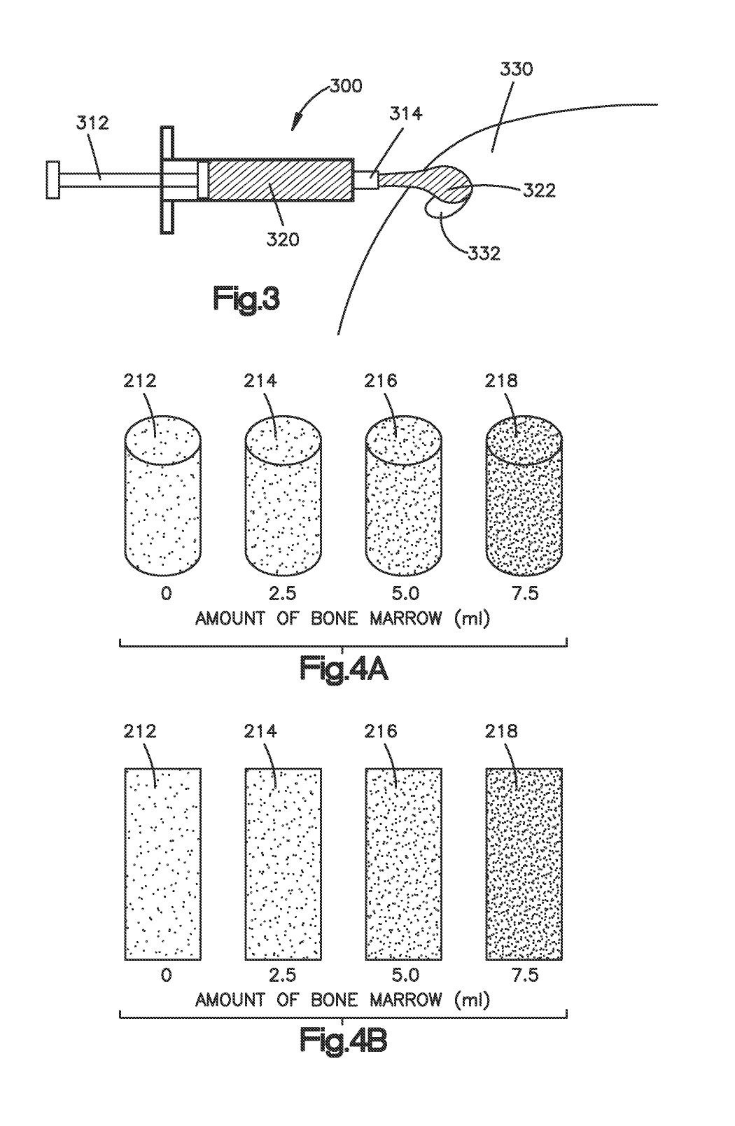

Referring now to FIG. 3, a delivery system 300 can be configured to deliver the bone cement 230 to the target location. For instance, the delivery system 300 can include a storage chamber 310 that contains a quantity of uncured bone cement 320 as described above. The storage chamber 310 can be in the form of a syringe, and the delivery system 300 can further include a plunger 312 that can be pressed to dispense the uncured bone cement 320 from the storage chamber 310 out through a nozzle 314. It should be appreciated that the delivery system 300 can include additional storage chambers or syringes that are operably coupled to the storage chamber 310, such that additional ingredients can be mixed with the uncured bone cement 320 prior to delivery of the uncured bone cement to the target location.

In one example, the uncured bone cement 320 is prepared just before a procedure from components such as liquid phase and particulate components as described in examples above. The uncured bone cement 320 is then applied and cured in place.

The bone cement 230 having one or more mechanical properties that match those of e.g. osteoporotic bone can be used in any suitable indication where bone is to be augmented, for instance at the proximal femur, the proximal humerus, long bones, vertebral bodies or the like.

As shown in the following example, the bone cement 230 exhibits a decrease in stiffness when compared to conventional bone cements that do not include the bone marrow. As discussed above, a reduced stiffness of the bone cement 230 compared to conventional bone cements efficiently reduces the risk that adjacent vertebral bodies will fracture due to vertebroplasty procedures.

In addition to the reduced stiffness, another property which is influenced by the addition of bone marrow is the maximum polymerization temperature of the exothermic polymerization of PMMA. Typically, polymerization of the PMMA can generate enough heat and increase the temperature of the bone cement to such a degree as to cause tissue necrosis. Because the bone cement 230 can include a lower content of monomer (MMA), which is the component that generates the heat during the polymerization reaction, compared to conventional bone cements, the maximum polymerization temperature can be lowered. In addition to the reduced monomer content, the bone marrow can act as a heat sink during the polymerization reaction, and thus further reduces the temperature in the bone cement during curing.

Thus, the bone cement 230 may be particularly desirable during cranial reconstruction where the bone cement may contact the delicate dura mater, tissues, and bone structures. As described above, the bone cement 230 can also be useful for vertebroplasty whereby the bone cement 230 has a mechanical property, such as Young's modulus and/or yield strength, that substantially matches that of the solid body to which the bone cement 230 is to adhere at the target location. Further more, the inclusion of bone marrow in the bone cement 230 can enhance healing through properties such as increased osteogeneration, increased osteoconductivity and increased osteoinductivity.

### Example

The following example was carried out using commercial PMMA cement commercially available in the Vertecem V+ kit from Synthes, having a place of business in West Chester, Pa. Vertecem V+ bone cement is a slow setting, radiopaque acrylic bone cement configured for use in a number of applications such as percutaneous vertebroplasty. The fluid phase is composed of 99.35% methyl-methacrylate (MMA), 0.65% N, N-dimethyl-p-toluidine as activator and very small quantities (60 ppm) of hydroquinones as a stabilizer. The polymer powder is composed of 44.6% PMMA, 0.4% benzoyl peroxide which initiates the polymerization, 40% zirconium dioxide as a radiopaque agent, and 15% hydroxyapatite as a radiopaque and bioactive agent.

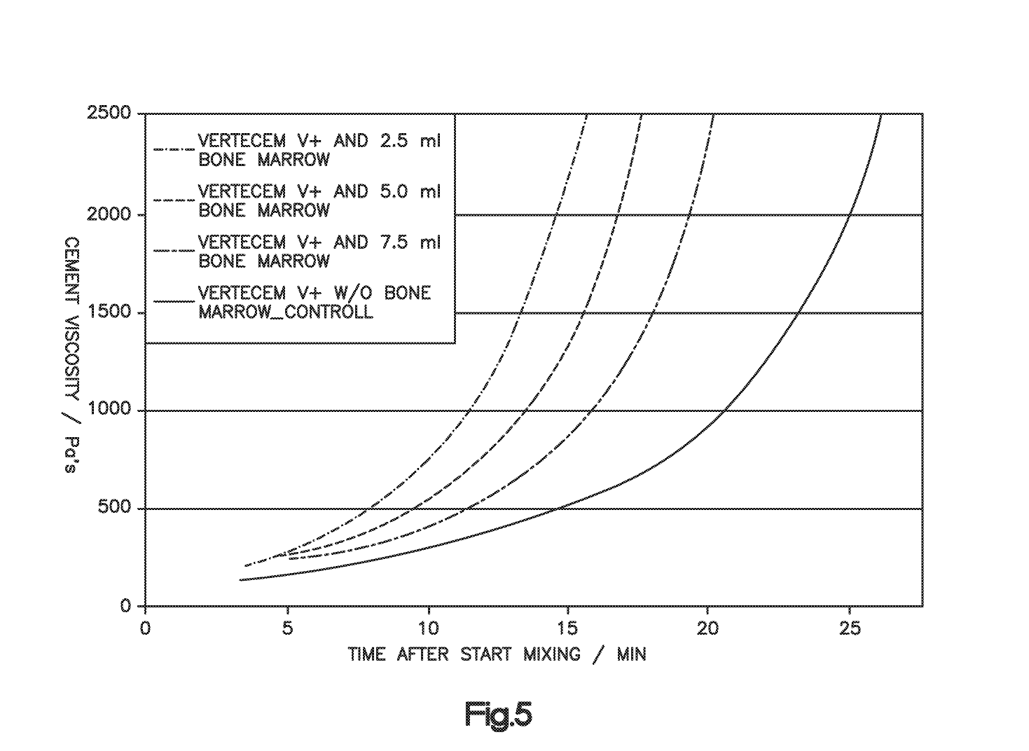

Bone marrow from the iliac crest was harvested from a sheep which underwent another surgery where bone marrow had to be removed. Four sample groups 212-218 of cement included varying quantities of bone marrow per batch of Vertecem V+ cement. Each batch of Vertecem V+ cement included a mixture of 26 g polymer powder per 10 ml MMA. Referring also to FIGS. 4A-B, the first sample group 212 (Group 1) was a control group devoid of bone marrow. The second sample group 214 (Group 2) included 2.5 ml of bone marrow. The third sample group 216 (Group 3) included 5 ml of bone marrow. The fourth sample group 218 (Group 4) included 7.5 ml of bone marrow.

More specifically, the liquid component of the Vertecem V+ cement was added to the powder component of the Vertecem V+ cement contained in the mixer, and the components were mixed for 10 seconds. Next, the above-identified quantities of freshly harvested bone marrow were added to Groups 2-4, respectively, followed by an additional mixing of all sample groups for 20-30 seconds, resulting in a substantially homogenous cement paste. Several tests were performed on four sample groups of cement provided as described above with respect to Groups 1-4.

To prepare the sample groups for mechanical testing, the paste of each sample group was filled into cylindrical Teflon molds (30 mm height, 10 mm diameter) and allowed to cure. Afterwards the hardened cylinders of the test sample groups were removed from the molds, and sawed and ground to the length of 20 mm each having parallel end surfaces. The resulting four sample groups 212, 214, 216, and 218 are illustrated in FIGS. 4A-B. Afterwards the sample groups 212, 214, 216, and 218 were subjected to for mechanical compression testing according to the ISO 5833 testing protocol. In particular, ten samples were tested for each sample group. Young's modulus and yield strength were determined for each sample of the four sample groups, and the results are plotted in Table 1.

TABLE 1
Mechanical Properties gathered from compression
test of the different material compositions (Mean ±
standard deviation, n = 10)

| | Bone | Young's | Yield |
|---|---|---|---|
| Group | Marrow | Modulus | Strength |
| number | Content | (Mpa) | (MPa) |
| 1 | 0 | 1833.16 ± 47.36 | 57.76 ± 3.88 |
| 2 | 2.5 | 1283.45 ± 74.75 | 37.18 ± 2.04 |
| 3 | 5 | 895.5 ± 76.10 | 26.02 ± 1.79 |
| 4 | 7.5 | 737.06 ± 81.97 | 23.02 ± 1.38 |

Table 1 shows that the Young's modulus and yield strength were inversely related to the amount of bone marrow of the sample groups. More specifically, the Young's modulus decreased from approximately 1830 MPa with 0% bone marrow content to approximately 740 MPa with 7.5 ml bone marrow content. Similarly, the yield strength decreased from approximately 58 MPa with 0% bone marrow content to approximately 23 MPa with 7.5 ml bone marrow content. Because the data was not normally distributed, a Kruskal Wallice test was used, and showed significant differences for both parameters (p≦0.001) between the sample groups. The mechanical properties of the sample groups can be compared to like mechanical properties of human cancellous bone, which have been reported as having a Young's modulus of 352±145 MPa and a yield strength of 2.5±1.5 MPa (mean±standard deviation) based on sixty-two male and female vertebral bodies. Due to high correlation between bone density (which was reported to be 0.17 g/cm³), values below the mean can be identified as osteoporotic bone.

The maximum cement temperature and setting time during curing were also measured for each sample group according to the ISO 5833 testing protocol. In particular, the cement paste of each of the four sample groups described above were injected into a Teflon mold (6 mm height, 60 mm diameter) and the temperature of the cement was measured using a TC-08 thermocouple data logger commercially available from PICO Technology, having a place of business in St. Neots, U.K. The temperature sensor was connected to a PC interface (PicoLog data acquisition software, commercially available from PICO Technology) for storing the data (the sampling rate was 1 Hz). The accuracy of the measurement was stated to be 0.5° C. by the manufacturer. The cement and the test equipment were maintained at 23±1° C. and at a relative humidity of not less than 40% at least 2 h before and during testing. Three samples for each sample group were measured, according to the ISO 5833 testing protocol. Setting time is defined as the time after start mixing the cement, when the temperature of the cement is midway between the ambient temperature and the peak temperature. Maximum temperature (Tₘₐₓ) and setting time (tₛₑₜ) are presented in Table 2 as mean±standard deviation

TABLE 2
Maximum temperatures and setting times
for all material compositions.

| | Quantity of bone marrow | | | |
|---|---|---|---|---|
| | 0 ml | 2.5 ml | 5.0 ml | 7.5 ml |
| Tₘₐₓ/° C. | 60.85 ± 2.5 | 60.7 ± 7.2 | 42.3 ± 3.2 | 38.0 ± 4.4 |
| tₛₑₜ/min | 28.3 ± 0.7 | 16.4 ± 3.3 | 20 ± 1 | 24.8 ± 1 |

Maximum temperature Tₘₐₓ setting time tₛₑₜ as mean ± standard deviation (n = 3) for the four sample groups.

Table 2 illustrates that the maximum temperature decreased on average from 60.85° C. for the control sample group devoid of bone marrow to 42.3° C. for the sample group containing 5 ml bone marrow, to 38° C. for the sample group containing 7.5 ml bone marrow. When 2.5 ml bone marrow was added, the maximum temperature was measured to be approximately 60.7° C. at the corresponding shortest setting time of 16.4 minutes.

The cement viscosity during curing was also measured for each sample group. In order to determine qualitatively the polymerization kinetic of cement hardening and the initial viscosity and the time reaching a viscosity of 2000 Pa*s quantitatively a rheological study was performed to derive the cement viscosity as function of time after starting cement preparation. For the viscosity measurements, 3 ml of the prepared cement of each of the sample groups was placed in a rotational rheometer (Viscosafe Viscometer, commercially available from Anton Paar GmbH, having a place of business in Graz, Austria). The real viscosity was recorded every 5 seconds directly to a PC using the corresponding software (RHEOPLUS/32 Mult±128 V2.66, commercially available from Anton Paar). The rheometer was set to operate at an oscillatory frequency of 1 Hz and a maximum torque of 3 Nm. The viscosity measurements were started at 3.5 min after start of mixing. The initial viscosity was determined as minimal viscosity measured during the rheological data acquisition. Three trials were performed for each of the four sample groups (0 ml, 2.5 ml, 5 ml and 7.5 ml bone marrow additive, respectively). The initial viscosities and times until a cement viscosity of 2000 Pa*s was reached for the various sample groups are presented as means and standard deviations (mean±SD). The cement viscosity as a function of time after the start of mixing is presented with one representative measurement for each ambient temperature.

Referring to FIG. 5, the cement viscosity is plotted as function of time for each of the sample groups after the start of mixing. No significant differences of initial viscosity was observed for the sample groups. In general, the hardening time to reach a cement viscosity of 2000 Pa·s was reduced as increasing amounts of bone marrow was present in the sample groups. The cement sample group containing 2.5 ml bone marrow presents the lowest hardening time, and the hardening time increased for sample groups containing increasing amounts of bone marrow. It is believed that the control group of bone cement devoid of bone marrow is associated with a high hardening time, and that the addition of a minimal amount of bone marrow to the bone cement causes the hardening time to be reduced with respect to the control group, and subsequent addition of bone marrow causes the hardening time to increase. Phase separation of the biphasic materials of the bone cement was neither observed during mixing nor during rheological data acquisition. Accordingly, the bone cement can include a substantially homogenous mixture of the materials that comprise the bone cement.

The materials, such as polymers, ceramic phases, and solvents shown are illustrated as examples only. Likewise, the specific preparation and test methods are shown as examples only.

Although various embodiments have been described in detail, it should be understood that various changes, substitutions, and alterations can be made herein without departing from the spirit and scope of the invention, for instance as defined by the appended claims. For example, the above-described embodiments and examples (or one or more aspects thereof) may be used in combination with each other. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, composition of matter, means, methods, or steps, presently existing or later to be developed that perform substantially the same function or achieve substantially the same result as the corresponding embodiments described herein may be utilized.

What is claimed is:

1. A cement configured to be introduced to a target location and allowed to cure, the cement comprising:
a monomer;
a polymerization initiator; and
a quantity of bone marrow in sufficient amount such that the cement has a mechanical property that substantially matches a like mechanical property of the target location.

2. The cement of claim 1, further comprising styrene to build into a polymer backbone.

3. The cement of claim 1, having a yield strength between approximately 5 MPa and approximately 60 MPa when the bone cement is cured.

4. The cement of claim 3, wherein the yield strength is between approximately 20 MPa and approximately 50 MPa when the bone cement is cured.

5. The cement of claim 1, having a Young's modulus of between 100 MPa and 1000 MPa when the bone cement is cured.

6. The cement of claim 5, wherein the Young's modulus is between approximately 200 MPa and approximately 500 MPa when the bone cement is cured.

7. The cement of claim 1, wherein the bone marrow comprises between 10-60% of the cement by volume.

8. The cement of claim 1, wherein the substantially matched mechanical property is a Young's modulus.

9. The cement of claim 1, wherein the substantially matched mechanical property is a yield strength.

10. A bone cement configured to be introduced into a target bone and allowed to cure, the bone cement comprising:
a quantity of methyl-methacrylate monomer;
a polymerization initiator configured to polymerize the monomer; and
bone marrow in an amount sufficient to provide a mechanical property of the bone cement that substantially matches a like mechanical property of the target bone.

11. The bone cement of claim 10, further comprising an activator that includes N, N-dimethyl-p-toluidine.

12. The bone cement of claim 11, further comprising a stabiliser that includes hydroquinone.

13. The bone cement of claim 10, wherein the polymerization initiator comprises benzoyl peroxide.

14. A bone cement configured to be introduced into a target bone and allowed to cure, the bone cement comprising:
a solid particulate component; and
bone marrow in an amount sufficient to provide a mechanical property of the bone cement that substantially matches a like mechanical property of the target bone.

15. The bone cement of claim 14, wherein the solid particulate component includes poly methyl methacrylate (PMMA) powder.

16. The bone cement of claim 15, wherein the solid particulate component includes hydroxyapatite.

17. The bone cement of claim 14, further including a radiopaque agent.

18. The bone cement of claim 17, wherein the radiopaque agent is barium sulphate.

19. The bone cement of claim 14, further comprising a quantity of a monomer, and a polymerization initiator configured to polymerize the monomer.

20. The bone cement of claim 15, wherein the monomer comprises methyl-methacrylate.

21. A bone cement configured to be introduced to a target location and allowed to cure, the cement comprising:
a polymer; and
a quantity of bone marrow in sufficient amount such that the cement has a mechanical property that matches a like mechanical property of the target location.

22. A method of fabricating a bone cement having a mechanical property, comprising:
determining a mechanical property of a target bone to which the mechanical property of the bone cement is to be substantially matched;
mixing a monomer with a polymerization initiator; and
adding a quantity of bone marrow to the mixture in sufficient amount so as to match the mechanical property of the bone cement with the mechanical property of the bone.

23. The method of claim 22, wherein the adding step further comprises creating the bone cement having a yield strength between approximately 5 MPa and approximately 60 MPa when the bone cement is cured.

24. The method of claim 23, wherein the adding step further comprises creating the bone cement having a yield strength between approximately 20 MPa and approximately 50 MPa when the bone cement is cured.

25. The method of claim 22, wherein the adding step further comprises creating the bone cement having a Young's modulus between approximately 100 MPa and approximately 1000 MPa.

26. The method of claim 25, wherein the adding step further comprises creating the bone cement having a Young's modulus between approximately 200 MPa and approximately 500 MPa.
